(19) United States
(12) Patent Application Publication
(10) Pub. No.: US 2016/0324446 A1
(43) Pub. Date: Nov. 10, 2016

(54) SYSTEM AND METHOD FOR DETERMINING NEURAL STATES FROM PHYSIOLOGICAL MEASUREMENTS

(71) Applicant: The General Hospital Corporation, Boston, MA (US)

(72) Inventors: Michael J. Prerau, Somerville, MA (US); Patrick L. Purdon, Somerville, MA (US); Emery N. Brown, Boston, MA (US)

(21) Appl. No.: 15/034,246

(22) PCT Filed: Nov. 5, 2014
(86) PCT No.: PCT/US2014/064144

§ 371 Date: May 4, 2016

Related U.S. Application Data
(60) Provisional application No. 61/900,084, filed on Nov. 5, 2013.

(51) Int. Cl. A61B 5/11; A61B 5/048; A61B 5/053; A61B 5/00

(52) U.S. Cl. CPC A61B 5/1106; A61B 5/4839; A61B 5/048; A61B 5/0533; A61B 5/4812; A61B 5/4821

(57) ABSTRACT

Systems and methods for identifying physiological states of a patient are provided. In one aspect, a method includes receiving a time-series of physiological data, and generating a multinomial regression model that includes regression parameters representing signatures of multiple neural states. The method also includes estimating probabilities for each of the neural states by applying the regression model to the time-series of physiological data, and identifying one of a current and future brain state of the patient using the estimated probabilities. The method further includes generating a report indicating a physiological state of the patient.

### CROSS-REFERENCE TO RELATED APPLICATIONS

The present application is based on, claims priority to, and incorporates herein by reference U.S. Provisional Application Ser. No. 61/900,084, filed Nov. 5, 2013, and entitled “DISCRETE STATE ESTIMATION FROM EEG AND OTHER PHYSIOLOGICAL DATA.”

### STATEMENT REGARDING FEDERALLY SPONSORED RESEARCH

This invention was made with government support under DP2 OD006454 awarded by the National Institutes of Health. The government has certain rights in the invention.

### BACKGROUND OF THE INVENTION

The present disclosure generally relates to systems and method for monitoring and controlling a state of a patient and, more particularly, to systems and methods for monitoring and/or controlling physiological states of a patient.

General anesthesia (“GA”) is a drug-induced, reversible condition manifested by hypnosis (loss of consciousness), amnesia (loss of memory), analgesia (loss of pain sensation), akinesia (immobility), and autonomic stability. Every day, in United States alone, over 100,000 patients depend on general anesthesia for the ability to undergo vital clinical procedures. During specific medical procedures, patients must be adequately anesthetized to prevent awareness or post-operative recall. Excessive dose administration, however, can delay emergence from anesthesia and could contribute to post-operative delirium or cognitive dysfunction. It is therefore important to be able to characterize and monitor clinically observable biomarkers of depth of anesthesia so that complications from over- or under-anesthetizing patients may be mitigated.

One such biomarker includes a phenomenon known as burst suppression, which is an example of an electroencephalogram (“EEG”) measurement pattern that consist of alternating epochs of electrical bursting activity, or bursts, and isoelectric periods of no appreciable electrical activity, or suppressions. These are manifested as a result of a patient's brain having severely reduced levels of neuronal activity, metabolic rate, and oxygen consumption. In particular, burst suppression is commonly observed in profound states of GA, where the period between burst epochs is dependent upon the dose of the anesthetic administered. One example of a profound state of a patient under general anesthesia is medical-induced coma. A variety of clinical scenarios require medical coma for purposes of brain protection, including treatment of uncontrolled seizures—status epilepticus—and brain protection following traumatic or hypoxic brain injury, anoxic brain injuries, hypothermia, and certain developmental disorders. Therefore, accurate characterization of burst suppression has broad range of applicability, including monitoring and controlling depth of anesthesia during specific medical procedures, as well as neuro-protective care.

The current clinical standard for evaluating burst suppression is through visual inspection of filtered EEG time-domain traces by a medical practitioner using a clinical definition of burst activity. However, visual scoring of burst suppression data in this manner is highly subjective, and can result in great variability in output between scorers. Several methods for automated tracking of burst suppression have been proposed, the majority of which involves computing an index for a specified EEG time-series using associated signal amplitudes, or energies. When the index crosses a specified threshold, the EEG is said to have transitioned into a burst or suppression state, depending of the direction of crossing. However, such methods are limited by the fact that they reduce the data to a single dimension, and rely on subjectively-defined thresholds that have no statistical interpretation. Consequently, these methods are unable to distinguish between bursts and high-amplitude motion artifacts, which occur frequently in clinical scenarios. Furthermore, these methods do not address the inter-dependence and temporal evolution of burst and suppression states, and could therefore produce physiologically implausible results.

Alternatively, machine-learning unsupervised classification techniques using support vector machine and hidden Markov model algorithms have been proposed for measuring pathological burst suppression detection in neonatal asphyxia. These methods use feature vectors derived from EEG data. While these methods address multi-dimensionality, the features used are predominantly statistical measures of time-domain distribution properties rather than physiologically motivated metrics. These methods also require manual removal of motion artifacts.

The above methodologies have several major drawbacks. First, they all pose the problem of burst suppression characterization in terms of binary classification in a feature-space. As such, results from these methods currently do not produce any degree of confidence in their classification, which is important in situations that involve clinical decision-making. Second, such methods address burst suppression detection in the time domain. However, demarcating burst onset and offset time in the time domain can be extremely difficult and variable between scorers, especially during periods of transitions into unconsciousness when the burst period is small.

In particular with respect to anesthesia-induced burst suppression, burst and suppression intervals can be much narrower, and in general more variable than those encountered in other settings, such as in the case of coma patients. Therefore, characterization of anesthesia-induced burst suppression can be particularly challenging. Moreover, artifacts are often prevalent in acquired EEG data due to an ongoing medical intervention or equipment utilized.

Therefore, considering the above, there continues to be a clear need for systems and methods to accurately quantify and monitor physiological patient states, such as a brain states associated with the administration of one or more anesthetic compound, as well as for controlling such patient states.

### SUMMARY OF THE INVENTION

The present disclosure overcomes drawbacks of previous technologies by providing systems and methods directed to identifying and tracking brain states of a patient. Specifically, a probabilistic framework is described for use in detecting neural states, such as burst suppression events associated with the administration of drugs having anesthetic properties or sleep. Using a multinomial logistic regression approach identifying the likelihood of competing models using acquired physiological data, probabilities of multiple neural states may be estimated and used to determine brain states of a patient. In addition, the present approach includes use of temporal continuity constraints in the state estimates in order to ensure that the generated results are physiologically realistic.

In some aspects, systems and methods described herein may be used to estimate burst, suppression, and artifact states from time-series EEG data. Specifically, the present disclosure recognizes that when time-series data is transformed into the frequency-domain, the resulting spectral structure may be utilized to differentiate between different neural states. For instance, by leveraging the observation that the spectral content between burst, suppression and artifact states differ, for example, for a patient undergoing anesthesia or sedation, more effective discrimination between neural states can be achieved.

In accordance with one aspect of the present disclosure, a method for identifying a physiological state of a patient is provided. The method includes receiving a time-series of physiological data, and generating a multinomial regression model that includes regression parameters representing signatures of multiple neural states. The method also includes estimating probabilities for each of the neural states by applying the regression model to the time-series of physiological data, and identifying one of a current and future brain state of the patient using the estimated probabilities. The method further includes generating a report indicating a physiological state of the patient.

In accordance with another aspect of the present disclosure, a system for identifying a physiological state of a patient is provided. The system includes at least one sensor configured to acquire time-series physiological data from a patient, and at least one processor configured to receive the acquired time-series of physiological data, and generate a multinomial regression model that includes regression parameters representing signatures of multiple neural states. The at least one processor is also configured to estimate probabilities for each of the neural states by applying the regression model to the time-series of physiological data, and identify one of a current and future brain state of the patient using the estimated probabilities. The at least one processor is further configured to generate a report indicating a physiological state of the patient.

In accordance with yet another aspect of the present disclosure, a method for identifying a brain state of a patient is provided. The method includes acquiring a time-series of physiological data, and producing frequency-domain data using signals associated with time segments in the time-series physiological data. The method also includes generating a multinomial regression model that includes regression parameters representing signatures of multiple neural states, and estimating probabilities for each of the neural states by applying the regression model to the frequency-domain data. The method further includes identifying a brain state of the patient using the estimated probabilities, and generating a report indicating a brain state of the patient.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The present invention will hereafter be described with reference to the accompanying drawings, wherein like reference numerals denote like elements.

FIG. 1A-B are schematic block diagrams of a physiological monitoring system.

FIG. 2 is a schematic block diagram of an example system for identifying and tracking brain states of a patient, in accordance with the present disclosure.

FIG. 3 is a flow chart setting forth the steps of a process in accordance with the present disclosure

FIG. 4 is an illustration of an example monitoring and/or control system in accordance with the present disclosure.

FIG. 5A-B are graphical depictions of example data in the frequency and time domain representations, illustrating burst suppression events experienced by a patient under administration of propofol.

FIG. 6 is a flow chart setting forth the steps of another process in accordance with the present disclosure.

FIG. 7 is a graphical illustration depicting time estimates of neural states determined in accordance with the present disclosure.

FIG. 8 is a graphical illustration depicting use of systems and methods, in accordance with the present disclosure, to determine probabilities of neural states for a patient undergoing anesthesia.

FIG. 9 is a graphical illustration depicting use of systems and methods, in accordance with the present disclosure, to determine probabilities of neural states for a patient during sleep.

### DETAILED DESCRIPTION

The present disclosure provide systems and methods that implement a statistically-principled approach to characterizing brain states of a patient using physiological data, such as electroencephalogram (“EEG”) data. Specifically, embodiments described herein allow for detection of discrete neural states, such burst, suppression states and artifacts, using a multinomial logistic regression approach in an manner that is automated and more objective than visual scoring of time-series data. In some aspects, use of frequency-domain information is described, recognizing that time-series data features, such as burst events, have an underlying oscillatory structure that may be more effectively used to characterize brain states of a patient. Such spectral signatures could be difficult to capture consistently with methods relying on time-domain data representations. As will be described, demonstrations of the efficacy of this approach are provided with respect to clinical EEG data acquired during operating room surgery with GA under propofol.

However, it is envisioned that methodology of the present disclosure is readily suitable to a wide range of applications, and particularly to any set of clinically or experimentally relevant physiological states. Specifically, systems and methods described herein may be utilized to determine and quantify any mutually-exclusive physiological states. Examples include neural states related to depth of anesthesia, such as drug effect on/offset, loss/return of consciousness, and deep anesthesia states, as well as sleep states, such as wake, REM, N1, N2, N3. Other applications afforded by the present disclosure include monitoring and/or controlling anesthesia, sedation, sleep pathologies, age identification, drug identification, and k-complex and spindle detection, and so forth. In addition, the approach described can also be extended to include non-EEG correlates, such as muscle activity, eye movement, cardiac activity, galvanic skin response, respiration, motion, behavior, blood oxygenation and so forth.

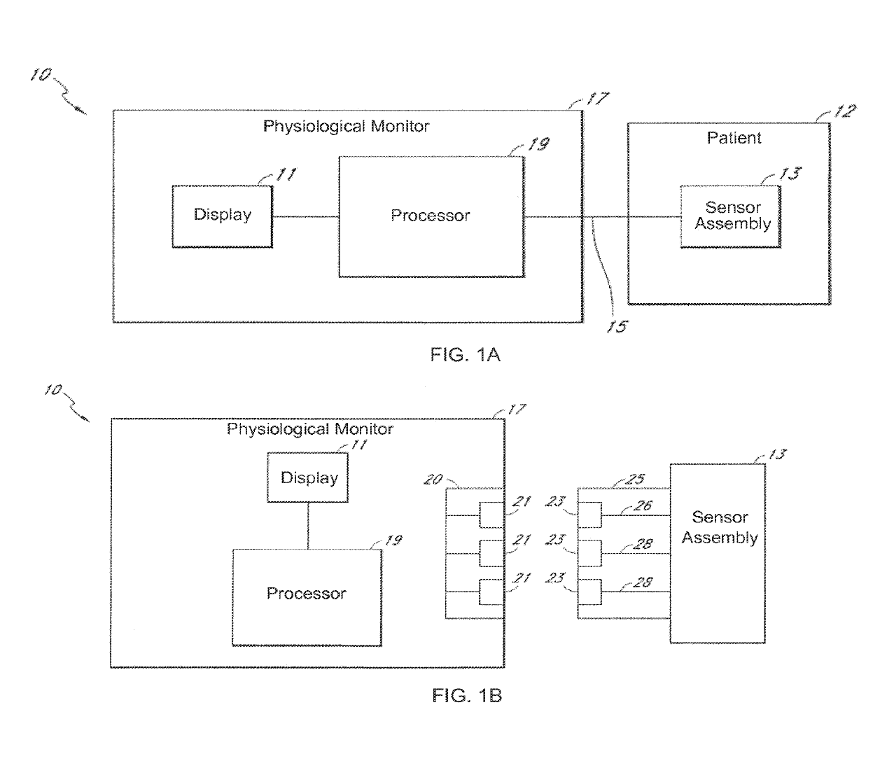

Referring specifically to the drawings, FIGS. 1A and 1B illustrate an example patient monitoring systems and sensors that can be used to provide physiological monitoring of a patient, such as consciousness state monitoring, with loss of consciousness or emergence detection.

For example, FIG. 1A shows an embodiment of a physiological monitoring system 10. In the physiological monitoring system 10, a medical patient 12 is monitored using a sensor assembly 13, which transmits signals over a cable 15 or other communication link or medium to a physiological monitor 17. The physiological monitor 17 includes a processor 19 and, optionally, a display 11. The sensor assembly 13 can generate respective physiological signals by measuring one or more physiological parameter of the patient 12. The signals are then processed by one or more processors 19, in accordance with the present disclosure. In some configurations, physiological monitor 17 may also include an input (not shown), configured to receive domain-specific information related to the monitored physiological parameters. The one or more processors 19 then communicate processed signals to the display 11 if a display 11 is provided. In an embodiment, the display 11 is incorporated in the physiological monitor 17. In another embodiment, the display 11 is separate from the physiological monitor 17. The monitoring system 10 is a portable monitoring system in one configuration. In another instance, the monitoring system 10 is a pod, without a display, and is adapted to provide physiological parameter data to a display.

For clarity, a single block is used to illustrate the sensor assembly 13 shown in FIG. 1A. It should be understood that the sensor assembly 13 shown can include one or more sensing elements such as, for example, electrical EEG sensors, oxygenation sensors, galvanic skin response sensors, respiration sensors, muscle activity sensors, and so forth, and any combinations thereof. In an embodiment, the sensor assembly 13 includes a single sensor of one of the types described. In another embodiment, the sensor assembly 13 includes at least two or more sensors. In each of the foregoing embodiments, additional sensors of different types are also optionally included. In addition, any combination of numbers and types of sensors are also suitable for use with the physiological monitoring system 10.

In some embodiments of the system shown in FIG. 1A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensors 13.

As shown in FIG. 1B, the sensor assembly 13 can include a cable 25. The cable 25 includes at least three conductors within an electrical shielding. One conductor 26 can provide power to a physiological monitor 17, one conductor 28 can provide a ground signal to the physiological monitor 17, and one conductor 28 can transmit signals from the sensor assembly 13 to the physiological monitor 17. For multiple sensors, additional conductors and/or cables can be provided.

In some embodiments, the ground signal is an earth ground, but in other embodiments, the ground signal is a patient ground, sometimes referred to as a patient reference, a patient reference signal, a return, or a patient return. In some embodiments, the cable 25 carries two conductors within an electrical shielding layer, and the shielding layer acts as the ground conductor. Electrical interfaces 23 in the cable 25 can enable the cable to electrically connect to electrical interfaces 21 in a connector 20 of the physiological monitor 17. In another embodiment, the sensor assembly 13 and the physiological monitor 17 communicate wirelessly.

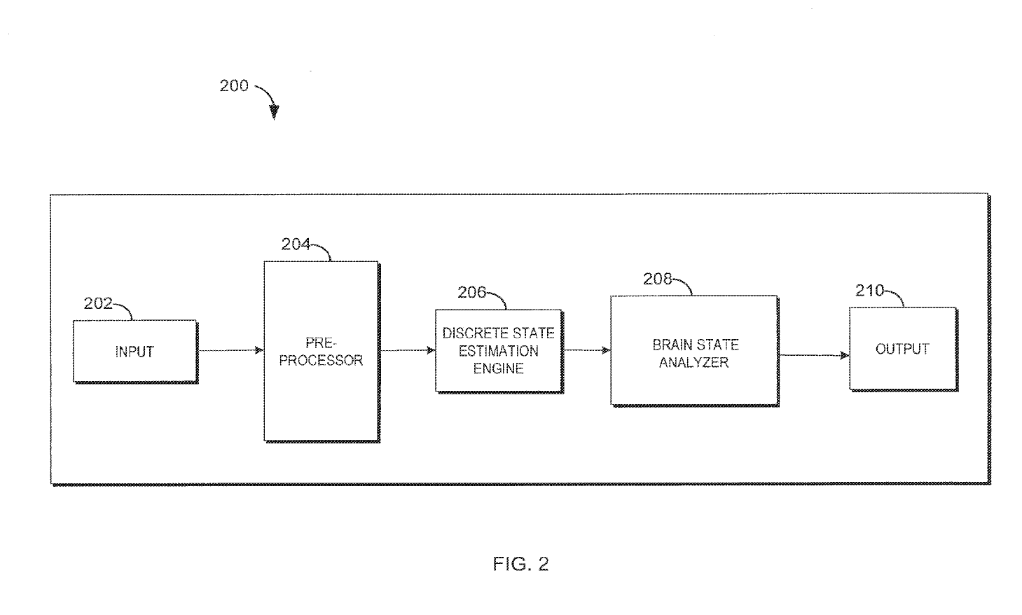

Referring to FIG. 2, an example system 200 for use in carrying out steps associated with determining a brain state of a patient using physiological data. The system 200 includes an input 202, a pre-processor 204, a discrete state estimation engine 206, a brain state analyzer 208, and an output 210. Some or all of the modules of the system 200 can be implemented by a physiological patient monitor as described above with respect to FIGS. 1A, and B.

The pre-processor 204 may be designed to carry out any number of processing steps for operation of the system 200. Specifically, the pre-processor 204 may be configured to receive and pre-process data or information received via the input 202. For instance, the pre-processor 204 may be configured to assemble a time-frequency representation of signals from time-series physiological data, such as EEG data, acquired from a patient and/or provided via input 202. In addition, the pre-processor 204 may configured to perform any desirable signal conditioning, such as filtering interfering or undesirable signals associated with the received physiological data. In some aspects, pre-processor 204 may be configured to provide other representations from time-series physiological data, including, for example, hypnograms, representing stages of sleep as a function of time.

In some aspects, the pre-processor 204 may also be capable of receiving instructions from a user, via the input 202. The addition, the pre-preprocessor 204 may also be capable of receiving patient or domain-specific information, for example, from a user or from a memory, database, or other electronic storage medium. For example, such information may be related to a particular patient profile, such as a patient's age, height, weight, gender, or the like, the nature of the medical procedure or monitoring being performed, including drug administration information, such as timing, dose, rate, anesthetic compound, and so forth. In addition, domain-specific information may include the nature or presence of specific states, or neural states, in regard to a patient and/or procedure, as well as knowledge related to the potential time evolution of such states. In some aspects, patient- and/or domain-specific information may be in the form of, or used to, determine regression parameters for a multinomial logistic regression model, for example, stored in a memory, database or other storage medium, and accessible by the pre-processor 204. Such parameters may be generated, for example, using training data acquired from a population and/or patient. In addition, the pre-processor 204 may be also configured to determine any or all of the above-mentioned patient and/or domain-specific information by processing physiological and other data provided via the input 202.

In some aspects, given multiple sets of potentially-observable brain states, pre-processor 204 may be configured to use a likelihood analysis to automatically determine which set of regression parameters fits the patient's data the best. For example, when monitoring general anesthesia for a patient with an unknown age, unknown medical history, and unknown current medications, it is possible to automatically determine which set of regression parameters should be used for that patent given the observed data.

In other aspects, regression parameters may be computed using additional custom brain states determined by a user. For example, if there is a particular brain state that a clinician observes during the monitoring of a patient during general anesthesia, the clinician could select examples of that data from the current record and create a custom brain state. The multinomial logistic regression parameters could be recomputed using data from the database along with the newly selected data, and a new set of parameters could be estimated incorporating the custom brain state.

In addition to the pre-processor 204, the system 200 may further include a discrete state engine 206, in communication with the pre-processor 202, designed to receive pre-processed physiological, and other data, as well as any patient or domain-specific information from the pre-processor 202, and using the data and information, carry out steps necessary for estimating probabilities of multiple, mutually-exclusive states associated with the patient. Specifically, as will be described, the discrete state engine 206 may be programmed to generate a multinomial logistic regression model using patient- and/or domain-specific parameters, as described, and using the model, estimate probabilities of specific physiological states, including neural states such as burst, suppression, or artifact states, observed during administration of anesthetic drugs or sleep.

Probabilities provided by the discrete state estimation engine 206 may then used by the brain state analyzer 208 to determine brain state(s) of a patient, such as states of consciousness, sedation, or sleep, along with confidence indications with respect to the determined state(s). Information related to the determined state(s) may then be relayed to the output 210, along with any other desired information, in any shape or form. In some aspects, the output 210 may include a display configured to provide, either intermittently or in real time, information, indicators or indices related to acquired and/or processed physiological data, determined neural state probabilities, determined brain states, and so forth.

In accordance with aspects of the present disclosure, a probabilistic framework is described herein for estimating discrete states from temporally evolving physiological data, such as EEG data. In this analysis, discrete time increments may be defined as

tₖ=kΔt (1)

where Δt is the time interval between each of the T observations, and k={1, . . . , T}. In some aspects, a frequency-domain representation of the data may be utilized. Specifically, a set F of fixed-interval frequency bins centered at

fⱼ=kΔf (2)

may be defined, where Δf is the frequency interval of each bin, and j={1, . . . , F}. Given a set of time-series EEG data that includes observations between times t₁ and t_{T}, and frequency bins centered at f₁ to f_{F}, a matrix F×T of frequency-domain observations may be constructed as follows

$$M = ( \begin{matrix}{m}_{1 , 1} & … & {m}_{1 , T} \\ ⋮ & ⋱ & ⋮ \\ {m}_{F , 1} & … & {m}_{F , T}\end{matrix} ) \qquad ( 3 )$$

where each element m_{i,j} represents a function of the power spectrum, such as magnitude, within frequency bin fᵢ at a time tⱼ.

Then, a set of Q mutually exclusive, discrete, states, S, may then be defined. By way of example, the following discussion considers burst, suppression and artifact neural states, where Q=3, and so

S={s_{burst},sₛᵤₚᵣₑₛₛᵢₒₙ,s_{artifact}} (4)

where s_{q} references the q^{th} element of S, and Sₖ represents the neural state at time tₖ. However, as mentioned, S can be defined to include any set of mutually-exclusive states, for example, by using patient- or domain-specific information.

As the only possible states are those in S, it follows that

$${\sum }_{q = 1}^{Q} Pr ( {S}_{k} = {s}_{k} ) = 1 \qquad ( 5 )$$

for any time point tₖ. It then follows that Ŝₖ, which is the predicted state at each time, is

$${\hat{S}}_{k} = {arg max}_{{s}_{c} ∈ S} [ Pr ( {S}_{k} = {s}_{c} ) ] . \qquad ( 6 )$$

In particular, given a set of EEG spectral observations during a period of burst suppression, the goal is to estimate Y, a Q×T matrix of temporarily evolving state probabilities

$$Y = ( \begin{matrix}Pr ( {S}_{1} = {s}_{burst} ) & … & Pr ( {S}_{T} = {s}_{burst} ) \\ Pr ( {S}_{1} = {s}_{supression} ) & … & Pr ( {S}_{T} = {s}_{supression} ) \\ Pr ( {S}_{1} = {s}_{artifact} ) & … & Pr ( {S}_{T} = {s}_{artifact} )\end{matrix} ) \qquad ( 7 )$$

The state probabilities may then be characterized using a multinomial logistic model of neural state probability of the form,

$$\begin{matrix}ln ( \frac{Pr ( {S}_{k} = {s}_{1} )}{Pr ( {S}_{k} = {s}_{Q} )} ) = {\overline{β}}_{1}^{T} {\overline{M}}_{k} \\ ⋮ \\ ln ( \frac{Pr ( {S}_{k} = {s}_{Q - 1} )}{Pr ( {S}_{k} = {s}_{Q} )} ) = {\overline{β}}_{Q - 1}^{T} {\overline{M}}_{k}\end{matrix} \qquad ( 8 )$$

where β is a F×(Q−1) matrix that includes model parameters, while β̅ᵢ and M̅ᵢ represent the i^{th} columns of the corresponding matrices. It then follows from Eqn. (5) that the probably at time tₖ is

$$Pr ( {S}_{k} = {s}_{q} ) = {exp ( {\overline{β}}_{q}^{T} {\overline{M}}_{k} ) [ 1 + {\sum }_{j = 1}^{Q - 1} exp ( {\overline{β}}_{j}^{T} {\overline{M}}_{k} ) ]}^{- 1} \qquad ( 9 )$$

for q<Q, and

$$Pr ( {S}_{k} = {s}_{Q} ) = {[ 1 + {\sum }_{j = 1}^{Q - 1} exp ( {\overline{β}}_{j}^{T} {\overline{M}}_{k} ) ]}^{- 1} \qquad ( 10 )$$

for q=Q. Therefore, in the case of a 3-state model, the state probabilities may be written as

$$Pr ( {S}_{k} = {s}_{burst} ) = {exp ( {\overline{β}}_{1}^{T} {\overline{M}}_{k} ) [ 1 + {\sum }_{j = 1}^{2} exp ( {\overline{β}}_{j}^{T} {\overline{M}}_{k} ) ]}^{- 1} \\ Pr ( {S}_{k} = {s}_{supression} ) = {exp ( {\overline{β}}_{2}^{T} {\overline{M}}_{k} ) [ 1 + {\sum }_{j = 1}^{2} exp ( {\overline{β}}_{j}^{T} {\overline{M}}_{k} ) ]}^{- 1} \\ Pr ( {S}_{k} = {s}_{artifact} ) = {[ 1 + {\sum }_{j = 1}^{2} exp ( {\overline{β}}_{j}^{T} {\overline{M}}_{k} ) ]}^{- 1} \qquad ( 11 )$$

In accordance with some aspects of the present disclosure, frequency-domain data may be produced using signals associated with acquired time-series physiological data. Specifically, frequency-domain data may be in the form of spectrograms generated, for example, from time-series EEG using a multitaper technique. In the case of the above-described 3-state model, to set up a regression, time segments representative of clear neural states, such as burst, suppression, and artifact states, may be identified in the spectrogram data. Then, for each identified segment, the median power spectrum may be computed, for example, and stored in the corresponding column in M. Since the neural state corresponding to each segment is known, a Y matrix can then be constructed such that the row corresponding to the scored state at each time has probability of 1 with the remaining elements 0. A parameter matrix β may then be estimated, for example, using an iteratively reweighted least squares algorithm to find the maximum a posteriori solution given the set of data captured in the M matrix, and the known states described in the Y matrix.

In a manner similar to the above, a domain-specific parameter matrix β may be obtained for any multinomial model that includes mutually-exclusive states using domain-specific data or information, for instance, provided by a user, retrieved from a database, memory or other storage medium, and/or determined from acquired physiological data, and so on.

Then, the above-domain specific parameter matrix β may be used to estimate the probability of the neural states given any newly observed physiological data, in accordance with Eqn. (11). The probabilities in turn can be used in Eqn. (6) to generate the state prediction, Ŝₖ.

In some aspects, information regarding the nature of the neural states may be used to inform the evolution of the probability estimates within the multinomial logistic regression. Such information could be used to construct priors on a state probability or construct a state transition matrix, which could be used in conjunction with the multinomial logistic regression. By including prior information into the state evolution, it is possible to render unrealistic transitions between states improbable. For example, it is unlikely that a patient can go from the state of burst-suppression to full wakefulness instantaneously. Thus, in this case, constructing a prior that makes the probability of wakefulness small given the fact that the current state is burst-suppression would prevent a transition that would not be possible for the patient.

Specifically, Q mutually-exclusive states {s₁, . . . , s_{Q}}, a state probability vector Pₖ at time tₖ may be defined as

$${P}_{k} = [ \begin{matrix}Pr ( {S}_{k} = {s}_{1} ) \\ ⋮ \\ Pr ( {S}_{k} = {s}_{Q} )\end{matrix} ] \qquad ( 12 )$$

It is then possible to impose constraints on the evolution of Pₖ in several ways. Specifically, in order to ensure that the generated probabilities and brain state estimates are physiologically reasonable, a continuity constraint in the temporal dynamics of the states may be imposed. For example, a maximum variability or change may be limited by a threshold quantity Δp between time points for each state's probability. That is, for each state s_{q} at each time tₖ, the state probability may be restricted such that

|Pr(Sₖ=s_{q})−Pr(Sₖ₋₁=s_{q})|≦Δp. (13)

State probabilities may then be renormalized so that the distribution sums to one. In addition, the prediction Ŝₖ may be further refined such that state transitions only occur when there is a high degree of certainty in Pr(Sₖ=s_{q}). Starting with the Eqn. (6) for the multinomial prediction of the state, let

$$\{ \begin{matrix}{\hat{S}}_{k} = {arg max}_{{s}_{c} ∈ S} [ Pr ( {S}_{k} = {s}_{c} ) ] & if {\hat{S}}_{k} \geq α \\ {\hat{S}}_{k} = {\hat{S}}_{k - 1} & otherwise\end{matrix} , \qquad ( 14 )$$

where α represents the desired confidence level. This can provide a statistically principled interpretation of the threshold used to detect states. Moreover, for example, bursts lasting less than a specified duration Bₘᵢₙ may be filtered out to make sure only physiologically plausible activity is extracted. For example, in one implementation, parameter values may be taken to be Δp=0.06, α=2/3, and Bₘᵢₙ=0.5 sec. Together, Eqns. (13) and (14) provide a computationally efficient approach of implementing a model of state temporal dynamics with a fixed continuity constraint as well as a state transition probability that is robust to noise.

In other aspects, it is possible to implement a specific model of state transition dynamics, which describes probability of each state at a given time given information from current or previous times. For example, a Markov model of transition probability could be implemented such that

Pₖ=FPₖ₋₁ (15)

where F is a Q×Q matrix of transition probabilities.

In yet some other aspects, it is possible to implement a specific model of state temporal dynamics, which describes the interrelationship between the states and time or other correlates. For example, Gaussian random walk models can be used model the temporal evolution of the states. In one implementation,

Pₖ=f(Pₖ₋₁) (16)

where f( ) can be any function of the input data, as well as hidden states

$${X}_{k} = [ \begin{matrix}{X}_{1} \\ ⋮ \\ {X}_{Q}\end{matrix} ] \qquad ( 17 )$$

which evolves according to a Gaussian random walk model, such that for each state x_{q},

xₖ^{q}=xₖ₋₁^{q}+ε_{q} (18)

where ε_{q}˜N(0,σ_{q}²). The state variance σ_{q}² may also be a function of time, input data, other states, or other correlates.

In some aspects, correlates of neural or physiological states could be used to inform other probability models relating behavioral or clinical states. For example, during general anesthesia, it could be useful to define the probability that a patient could be aroused to consciousness in response to a nociceptive stimulus. This ability to be aroused to consciousness is a function of the brain state. Thus, the probability of arousal may be modeled as a function of the patient's estimated brain state probabilities. For any set of J clinical or behavior states, {c₁, . . . , c_{J}}, the probability that the clinical or behavioral state Cₖ at time tₖ, is a given state cⱼ may be defined as

$$Pr ( {C}_{k} = {c}_{j} ) = {\sum }_{q = 1}^{Q} Pr ( {C}_{k} = {c}_{j} \text{\textbar} {S}_{k} = {s}_{q} ) Pr ( \text{\textbar} {S}_{k} = {s}_{q} ) , \qquad ( 19 )$$

where Pr(Cₖ=cⱼ|Sₖ=s_{q}) can be any function of the input data, the brain states, other clinical or behavioral states, or other correlates.

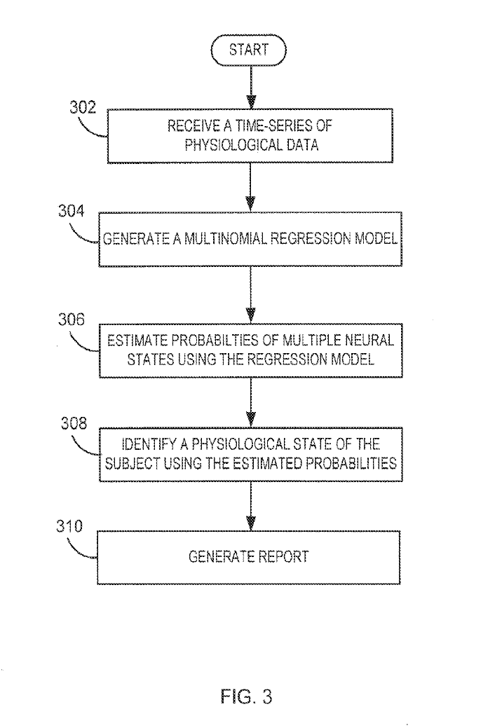

Referring now to FIG. 3, steps in an example process 300 for identifying physiological states of a patient, in accordance the present disclosure, are shown. Specifically, process 300 may begin at process block 302 by receiving a time-series of physiological data. In some aspects, such physiological data can be acquired, assembled, and pre-processed at process block 302, for example, using systems as described with reference to FIGS. 1 and 2. For instance, frequency-domain data may be produced using signals obtained from time segments associated with the received physiological data. Non-limiting examples of physiological data include EEG data, muscle activity data, eye movement data, electrocardiogram data, galvanic skin response data, respiration data, blood oxygenation data, motion data, behavioral data, drug data, and so on.

At process block 304, a multinomial regression model may then be generated, where the model includes regression parameters representing signatures of multiple neural states As mentioned, this can include receiving patient-specific or domain-specific information from a user, database, or other storage medium, and/or determining any or all patient- or domain-specific information from data acquired from the patient. In some aspects, parameters used to estimate the brain state probabilities could be selected or estimated based on patient information such as drug administration information, the age, gender, height, or weight of the patient, for instance, or the patient's prior medical history, including co-existing neurological or psychiatric disease, medication history, and other co-morbidities such as alcoholism. In addition, a received or determined domain-specific parameter set, representative of signatures for a number of mutually-exclusive states, may be utilized to generate the multinomial regression model at process block 304.

Then, at process block 306, probabilities for multiple states may be estimated, as outlined above, either intermittently or in real time. As described, this may include estimating probabilities for patient- or domain-specific mutually-exclusive or neural states, such as those associated with burst, burst suppression or noise activity experienced during administration of anesthesia or sleep. In accordance with aspects of the present disclosure, the temporal dynamics of the probabilities from process block 306 may be determined using one or more pre-determined or provided conditions, constraints or thresholds. As described, this can ensure physiologically accurate results.

As indicated by process block 308, using the estimated probabilities, present and/or future physiological states of a patient may then identified in accordance with Eqn. 6. For example, determined physiological states can include brain states exhibited during anesthesia or sleep. In some aspects, confidence levels, as described by Eqn. 13, may be included in identifying such physiological states. In some aspects, indices related to the identified physiological states, for example, states of consciousness or sleep, may also be computed at process block 308.

Then at process block 310 a report may be generated, of any form, either intermittently, or in real time. For example, the report may be provided via a display and include any patient or domain-specific information, as well as information related estimated probabilities mutually-exclusive or neural states, for instance, as wave-forms, as well as information related to identified physiological states, for instance, in the form of computed indices.

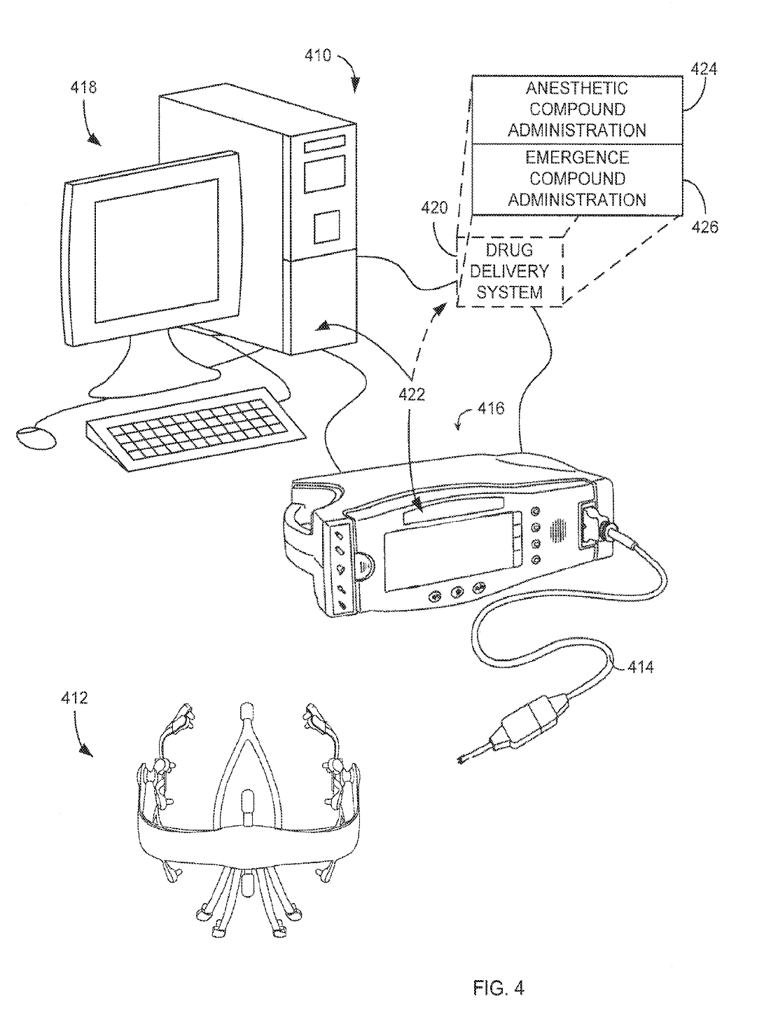

Referring to FIG. 4, a system 410 in accordance with one aspect the present invention is illustrated. The system 410 includes a patient monitoring device 412, such as a physiological monitoring device, illustrated in FIG. 4 as an electroencephalography (EEG) electrode array. However, it is contemplated that the patient monitoring device 412 may also include mechanisms for monitoring other physiological signals, such as galvanic skin response (GSR), for example, to measure arousal to external stimuli or other monitoring system such as cardiovascular monitors, including electrocardiographic and blood pressure monitors, and also ocular Microtremor monitors, and so on. One specific configuration of this design utilizes a frontal Laplacian EEG electrode layout with additional electrodes to measure GSR and/or ocular microtremor. Another configuration of this design incorporates a frontal array of electrodes that could be combined in post-processing to obtain any combination of electrodes found to optimally detect the EEG signatures described earlier, also with separate GSR electrodes. Another configuration of this design utilizes a high-density layout sampling the entire scalp surface using between 64 to 256 sensors for the purpose of source localization, also with separate GSR electrodes.

The patient monitoring device 412 is connected via a cable 414 to communicate with a monitoring system 416. Also, the cable 414 and similar connections can be replaced by wireless connections between components. As illustrated, the monitoring system 416 may be further connected to a dedicated analysis system 418. Also, the monitoring system 416 and analysis system 418 may be integrated.

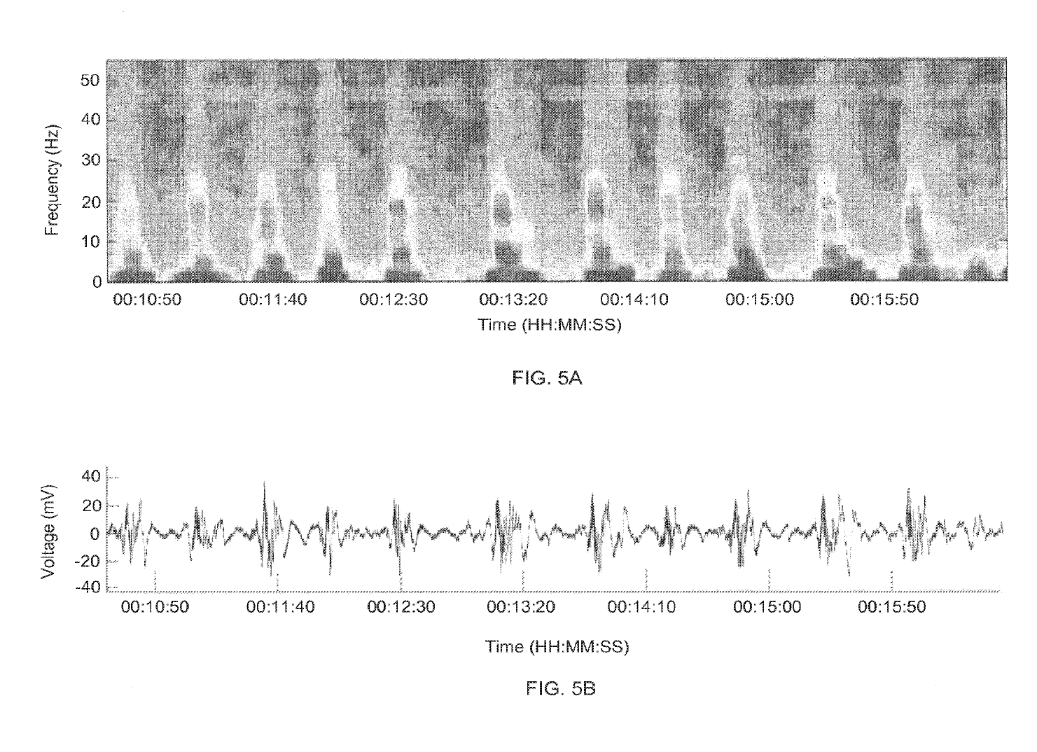

The monitoring system 416 may be configured to receive raw physiological signals acquired using the patient monitoring device 412 and assemble, and even display, the signals as raw or processed waveforms. Accordingly, the analysis system 418 may receive the waveforms from the monitoring system 416 and, process the waveforms and generate a report, for example, as a printed report or, preferably, a real-time display of information. By way of example, FIGS. 5A and B show frequency-domain and time-domain representations of burst suppression of a patient under administration of propofol. In some aspects, monitoring system 416 may determine patient- or domain-specific information using acquired and/or processed physiological signals. However, it is also contemplated that the functions of monitoring system 416 and analysis system 418 may be combined into a common system.

In some aspects, the analysis system 418 may be configured to determine a current and future brain state of a patient, in accordance with aspects of the present disclosure. That is, analysis system 418 may be configured to apply a probabilistic framework for use in detecting the likelihood of mutually-exclusive states, such as neural states associated with burst suppression or artifact events. Specifically, using a multinomial logistic regression model probabilities of multiple neural states may be determined and used by analysis system 418 to identify brain states of a patient, for example, during anesthesia or sleep. In some aspects, analysis system 418 may be configured to receive and utilize in the above analysis patient- or domain-specific information, for example, provided by a user, or obtained from a database, or other storage medium.

In some implementations, the system 410 may also include a drug delivery system 420. The drug delivery system 420 may be coupled to the analysis system 418 and monitoring system 416, such that the system 410 forms a closed-loop monitoring and control system. Such a closed-loop monitoring and control system in accordance with the present invention is capable of a wide range of operation, but includes user interfaces 422 to allow a user to configure the closed-loop monitoring and control system, receive feedback from the closed-loop monitoring and control system, and, if needed, reconfigure and/or override the closed-loop monitoring and control system.

In some configurations, the drug delivery system 420 is not only able to control the administration of anesthetic compounds for the purpose of placing the patient in a state of reduced consciousness influenced by the anesthetic compounds, such as general anesthesia or sedation, but can also implement and reflect systems and methods for bringing a patient to and from a state of greater or lesser consciousness.

For example, in accordance with one aspect, methylphenidate (MPH) can be used as an inhibitor of dopamine and norepinephrine reuptake transporters and actively induces emergence from isoflurane general anesthesia. MPH can be used to restore consciousness, induce electroencephalogram changes consistent with arousal, and increase respiratory drive. The behavioral and respiratory effects induced by methylphenidate can be inhibited by droperidol, supporting the evidence that methylphenidate induces arousal by activating a dopaminergic arousal pathway. Plethysmography and blood gas experiments establish that methylphenidate increases minute ventilation, which increases the rate of anesthetic elimination from the brain. Also, ethylphenidate or other agents can be used to actively induce emergence from isoflurane, propofol, or other general anesthesia by increasing arousal using a control system, such as described above. For example, the following drugs are non-limiting examples of drugs or anesthetic compounds that may be used with the present invention: Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and the like, as well as Zolpidem, Suvorexant, Eszopiclone, Ramelteon, Zaleplon, Doxepine, Diphenhydramine, and so on.

Therefore, a system, such as described above with respect to FIG. 4, can be provided to carry out active emergence from anesthesia by including a drug delivery system 420 with two specific sub-systems. As such, the drug delivery system 420 may include an anesthetic compound administration system 424 that is designed to deliver doses of one or more anesthetic compounds to a patient and may also include a emergence compound administration system 426 that is designed to deliver doses of one or more compounds that will reverse general anesthesia or the enhance the natural emergence of a patient from anesthesia.

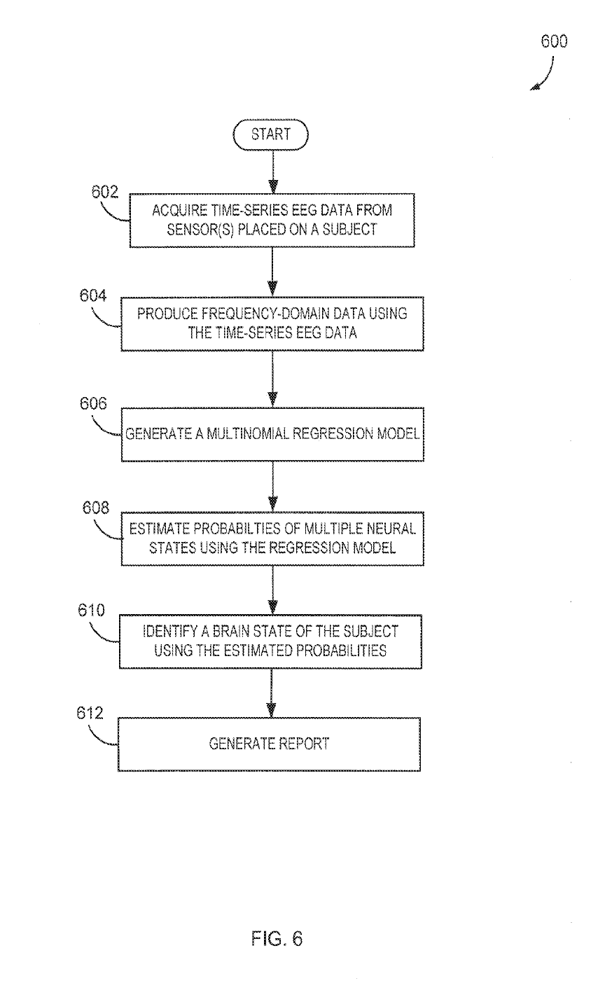

Referring to FIG. 6, steps of another example process 600 for identifying brain states of a patient are shown. In some aspects, process 600 may be carried out, for example, using a system as described with reference to FIG. 4. Specifically, process 600 may begin at process block 602 by acquiring a EEG data, as well as other physiological data. Non-limiting examples of other physiological data include muscle activity data, eye movement data, electrocardiogram data, galvanic skin response data, respiration data, blood oxygenation data, motion data, behavioral data, drug data, and so on. In some aspects, acquired EEG data may be pre-processed or conditioned at process block 602. For instance, acquired EEG data can assembled in the form of time-series data, from which frequency-domain data may be produced using signals obtained from time segments associated with the time-series data, as indicated by process block 604.

At process block 606 a multinomial regression model may then be generated using frequency-domain data, in accordance with aspects of the present disclosure. As described, the regression model may be generated using provided or determined patient-specific or domain-specific information, indicating at least the nature and number of mutually-exclusive neural states, for example, via provided or determined model parameters. Using the model, probabilities of multiple neural states may be estimated at process block 608, which may be utilized to identify a brain state of the patient, as indicated by process block 610. At process block 612, a report may be generated, of any shape or form.

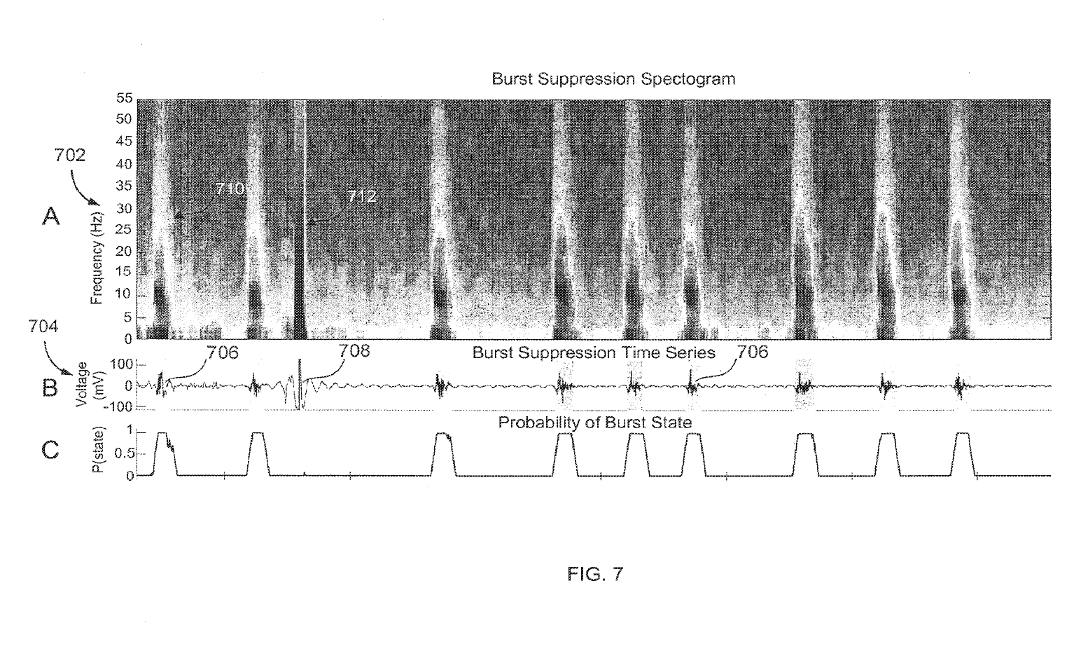

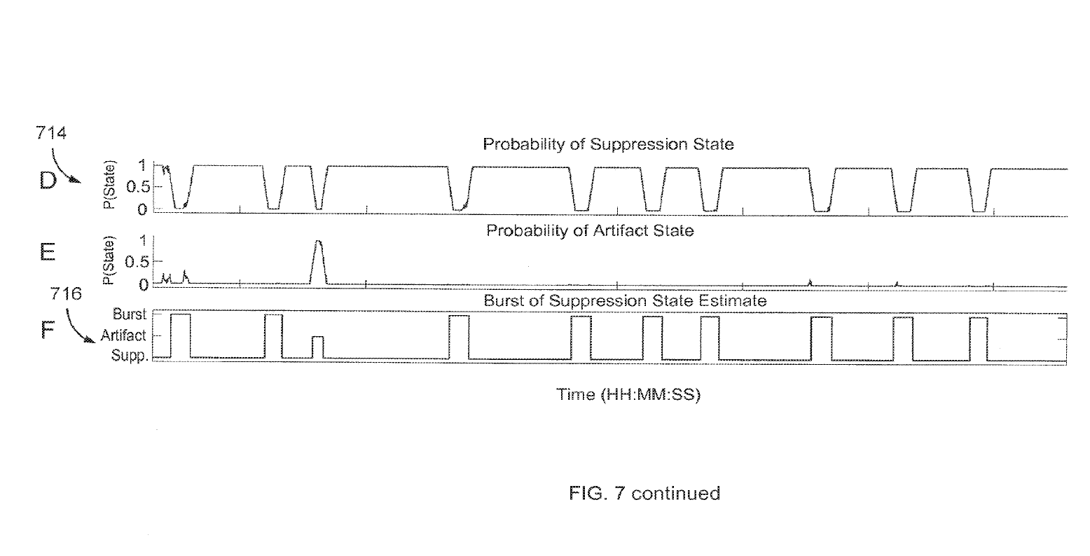

By way of example, an output generated, in accordance with aspects of the present disclosure, using EEG data obtained from a patient during administration of propofol is shown in FIG. 7. The spectrogram 702 was computed from the EEG time-series 704, and was visually scored, as indicated by regions of burst 706 and artifact 708 signals. As described, such visual scoring may utilized to determine patient or domain specific information.

In the spectrogram 702, bursts show a broadband frequency structure, with modes in the slow/delta and alpha bands, as indicated generally by 710. This structure is distinct from artifacts, which have a structure that includes high power at all frequencies, as indicated generally by 712. From the frequency-domain EEG data, neural state probabilities generally indicated at 714 were estimated from the multinomial logistic regression using methods, as described. From the probabilities, brain states 716, namely, Ŝₖ={s_{burst},sₛᵤₚᵣₑₛₛᵢₒₙ,s_{artifact}}, were then identified at multiple points in time, illustrating periods of burst, artifact and burst suppression during administration of propofol for this patient.

As shown in FIG. 7, the methodology described herein is able to distinguish clearly between bursts, suppression, and artifact periods. Specifically, these, and other data, show that the present approach is able to use frequency-domain information to automatically detect burst and suppression events in a manner that agrees closely with time-domain visual scoring.

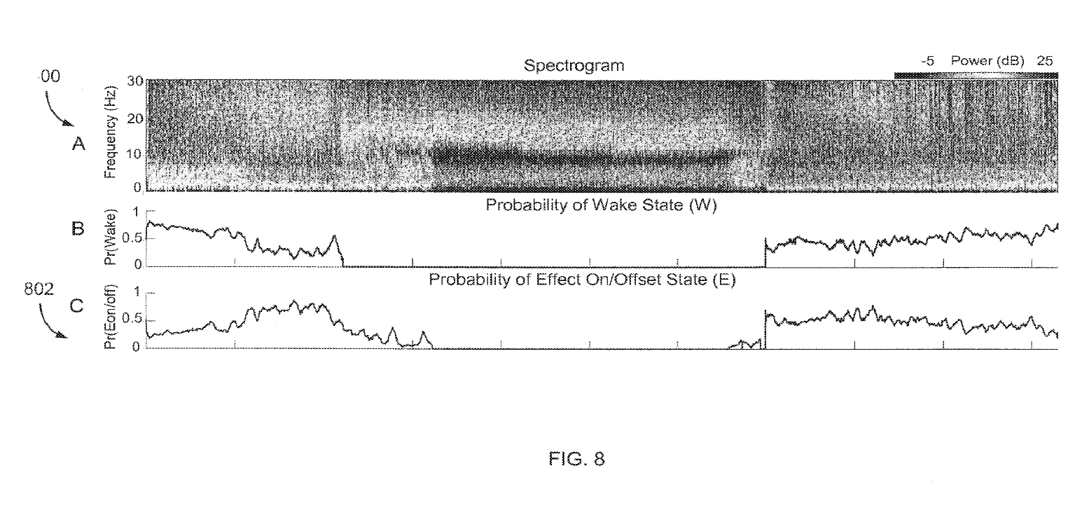

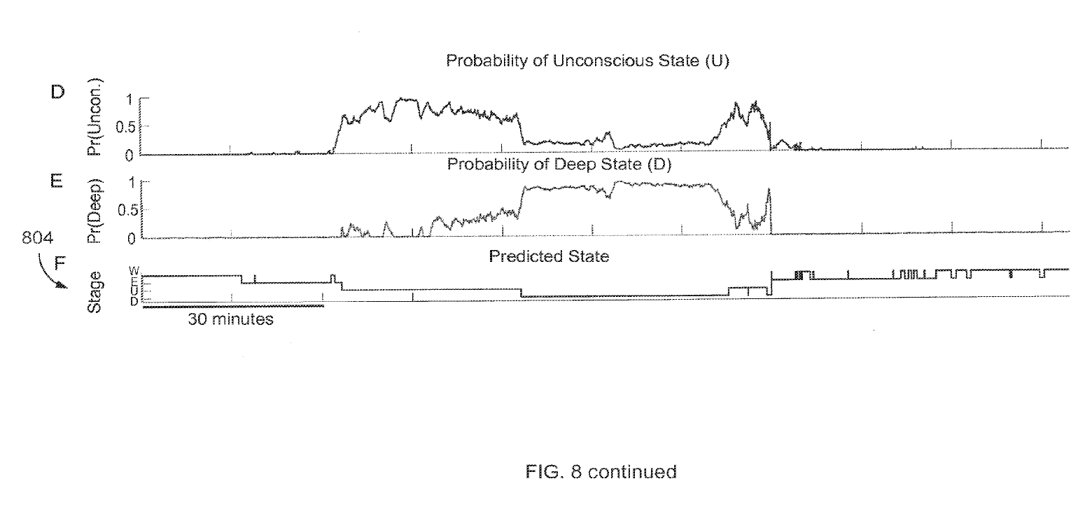

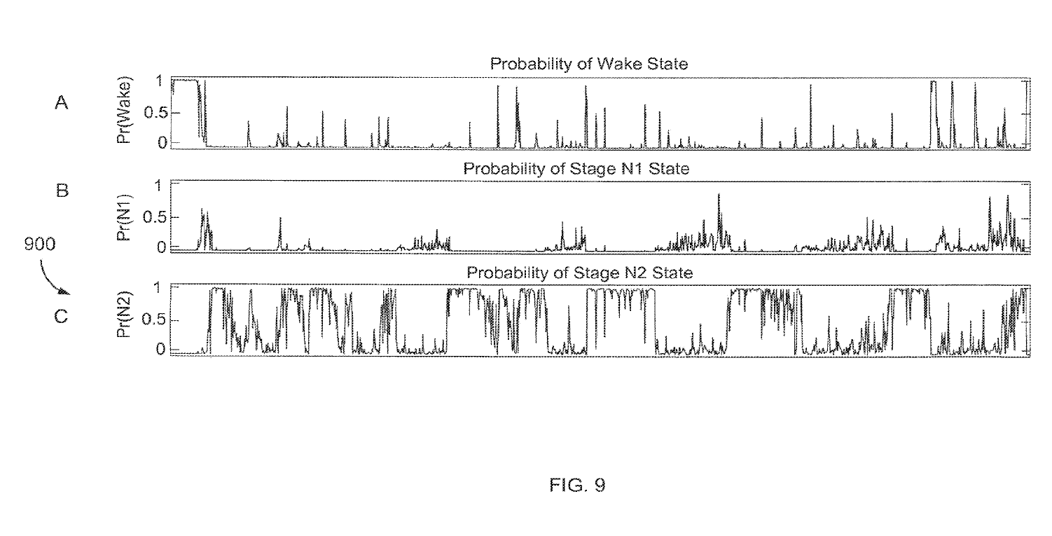

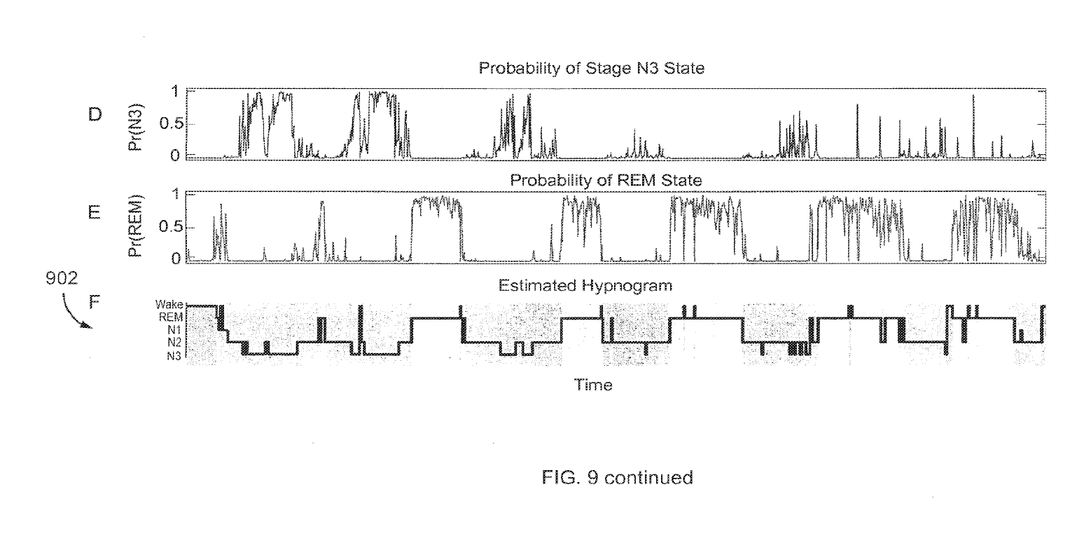

Systems and methods described herein may find use in a variety of other applications. Specifically referring to FIG. 8, an example is given with respect to spectrogram data 800 acquired during administration of anesthesia. As indicated generally by 802, time variation of probabilities for several neural states were estimated, including states of wake, effect On/Offset, unconscious, and deep, from which physiological states were identified, as indicated by 804. Similarly, as illustrated in FIG. 9, various the probabilities 900 for various stages of sleep, including wake, REM, N1, N2, N3, were also be estimated, using systems and methods described herein, to generate a hypnogram, as generally indicated by 902.

In some applications, systems and methods, as provided by the present disclosure, may be used to provide patient monitoring in intensive care situations and settings, where patients can be in a burst suppression brain state for a variety of reasons. For example, post-anoxic coma patients often remain in burst suppression during coma. Also, patients with epilepsy or traumatic brain injuries can be placed in medically-induced coma using general anesthetic drugs such as propofol. Changes in burst-induced hemodynamic or metabolic responses could indicate improving or declining brain health, and could prompt clinical intervention, or guide prognosis. By estimating the probability with which the patient is the burst and suppression states using the methods as provided by the present disclosure, it would be possible to more accurately compute metrics relating to the degree in which the subject is in burst-suppression, which could be used for drug control or to determine clinical intervention.

In some applications, systems and methods, as provided by the present disclosure, may be used to provide patient monitoring in operating room or intensive care settings, where patients undergo general anesthesia or sedation. For example, monitoring brain states during general anesthesia in the operating room is important for assessing when a patient is ready for surgery to begin and to make sure that a patient is neither over- nor under-anesthetized. By estimating the probability of different anesthesia-induced brain states using the methods provided by the present disclosure, would be possible to provide continuous monitoring or control of anesthetic drugs throughout a surgical procedure. Likewise, during intensive care scenarios, the patent is often placed under sedation for extended periods of time. By estimating the probability of different brain states associated with sedation using the methods provided by the present disclosure, it would be possible to provide continuous monitoring or control of sedative drugs throughout a patient's stay in an intensive care unit, thereby avoiding over-sedation, which has been linked to higher rates of mortality and delirium.

In other applications, systems and methods, as provided by the present disclosure, may be used to provide monitoring of sleep in clinical or home monitoring scenarios. For example, monitoring of sleep is important in clinical assessments of sleep apnea. As provided by the present disclosure, a real-time monitoring of sleep, or for post-hoc analysis of sleep stages can be performed. In addition, systems and methods herein could be used to characterize the efficacy of sleep therapeutic interventions, such as sleep medications. The present approach could also be used to monitor level or arousal and wakefulness to assess suitability for operation of heavy machinery, fine motor control, or other critical occupational requirements.

The approach of the present disclosure could also be used to identify and characterize brain states associated with psychiatric or neurological illness, and to characterize brain states induced by drugs intended to treat those illnesses. In addition, systems and methods described herein could be used to identify the effects of neuro-active drugs, including therapeutic drugs, or drugs of abuse such as alcohol, cocaine, ketamine, marijuana, or heroin. The monitoring could be used to identify therapeutically desired doses in medical applications. It could also be used to characterize levels of drug intoxication for purposes of cognitive and motor assessment.

In applications involving operating room and intensive care unit, the estimates of brain state probabilities could be used to annotate or visually guide EEG displays that clinicians use to manage patient brain states. In other applications, the present approach could be used to automatically identify artifacts within brain recordings, such as those induced by movement, clinical intervention, muscle activity, eye movement, bad electrode connections, or interference from other clinical instruments such as electrocautery.

The various configurations presented above are merely examples and are in no way meant to limit the scope of this disclosure. Variations of the configurations described herein will be apparent to persons of ordinary skill in the art, such variations being within the intended scope of the present application. Features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The patient matter described herein and in the recited claims intends to cover and embrace all suitable changes in technology.

1. A method for identifying a physiological state of a patient, the method comprising:
receiving a time-series of physiological data;
generating a multinomial regression model that includes regression parameters representing signatures of multiple neural states;
estimating probabilities for each of the neural states by applying the regression model to the time-series of physiological data;
identifying one of a current and future brain state of the patient using the estimated probabilities; and
generating a report indicating a physiological state of the patient.

2. The method of claim 1, wherein the time series of physiological data includes electroencephalogram (EEG) data.

3. The method of claim 1, the method further comprising acquiring the time-series of physiological data during administration of an anesthetic or during sleep.

4. The method of claim 1, the method further comprising producing frequency-domain data using signals associated with time segments in the time-series physiological data.

5. The method of claim 1, wherein the neural states are mutually-exclusive states.

6. The method of claim 1, the method further comprising obtaining at least one of patient-specific information or domain-specific information related to the different neural states.

7. The method of claim 6, the method further comprising determining the multiple neural states by using at least one of the patient-specific information and domain-specific information received.

8. The method of claim 1, wherein the neural states include a burst state, a burst suppression state, and an artifact state.

9. The method of claim 1, wherein the neural states include a wake state, an effect on/off state, an unconscious state and a deep state.

10. The method of claim 1, wherein the neural states include a wake state, a REM state, an N1 state, an N2 state, an N3 state.

11. The method of claim 1, the method further comprising applying an iteratively reweighted least squares technique to determine the regression parameters.

12. The method of claim 1, the method further comprising applying a continuity constraint to estimate temporal dynamics of estimated probabilities.

13. The method of claim 1, the method further comprising determining the regression parameters by applying a likelihood analysis using the time-series of physiological data.

14. A system for identifying a physiological state of a patient, the method comprising:
at least one sensor configured to acquire time-series physiological data from a patient;
at least one processor configured to:
receive the acquired time-series of physiological data;
generate a multinomial regression model that includes regression parameters representing signatures of multiple neural states;
estimate probabilities for each of the neural states by applying the regression model to the time-series of physiological data;
identify one of a current and future brain state of the patient using the estimated probabilities; and
generate a report indicating a physiological state of the patient.

15. The system of claim 14, wherein the time series of physiological data includes electroencephalogram (EEG) data.

16. The system of claim 14, wherein the at least one processor is further configured to acquire the time-series of physiological data during administration of an anesthetic or during sleep.

17. The system of claim 14, wherein the at least one processor is further configured to produce frequency-domain data using signals associated with time segments in the time-series physiological data.

18. The system of claim 14, wherein the neural states are mutually-exclusive states.

19. The system of claim 14, wherein the at least one processor is further configured to obtain at least one of patient-specific information or domain-specific information related to the different neural states.

20. The system of claim 19, wherein the at least one processor is further configured to determine the multiple neural states by using at least one of the patient-specific information and domain-specific information received.

21. The system of claim 14, wherein the neural states include a burst state, a burst suppression state, and an artifact state.

22. The system of claim 14, wherein the neural states include a wake state, an effect on/off state, an unconscious state and a deep state.

23. The system of claim 14, wherein the neural states include a wake state, a REM state, an N1 state, an N2 state, an N3 state.

24. The system of claim 14, wherein the at least one processor is further configured to apply an iteratively reweighted least squares technique to determine the regression parameters.

25. The system of claim 14, wherein the at least one processor is further configured to apply a continuity constraint to estimate temporal dynamics of estimated probabilities.

26. The system of claim 14, wherein the at least one processor is further configured to determine the regression parameters by applying a likelihood analysis using the time-series of physiological data.

27. A method for identifying a brain state of a patient, the method comprising:
acquiring a time-series of physiological data;
producing frequency-domain data using signals associated with time segments in the time-series physiological data;
generating a multinomial regression model that includes regression parameters representing signatures of multiple neural states;
estimating probabilities for each of the neural states by applying the regression model to the frequency-domain data;
identifying a brain state of the patient using the estimated probabilities; and
generating a report indicating a brain state of the patient.

28. The method of claim 27, wherein the time series of physiological data includes electroencephalogram (EEG) data.

29. The method of claim 27, the method further comprising acquiring the time-series of physiological data during administration of an anesthetic or during sleep.

30. The method of claim 27, wherein the neural states are mutually-exclusive states.

31. The method of claim 27, the method further comprising obtaining at least one of patient-specific information or domain-specific information related to the different neural states.

32. The method of claim 31, the method further comprising determining the multiple neural states by using at least one of the patient-specific information and domain-specific information received.

33. The method of claim 27, wherein the neural states include a burst state, a burst suppression state, and an artifact state.

34. The method of claim 27, wherein the neural states include a wake state, an effect on/off state, an unconscious state and a deep state.

35. The method of claim 27, wherein the neural states include a wake state, a REM state, an N1 state, an N2 state, an N3 state.

36. The method of claim 27, the method further comprising applying an iteratively reweighted least squares technique to determine the regression parameters.

37. The method of claim 27, the method further comprising applying a continuity constraint to estimate temporal dynamics of estimated probabilities.

38. The method of claim 27, the method further comprising determining the regression parameters by applying a likelihood analysis using the time-series of physiological data.
